(19) United States
(12) Patent Application Publication
(10) Pub. No.: US 2026/0249235 A1
(43) Pub. Date: Aug. 27, 2026

(54) INTEGRATION OF ETHANOL PRODUCTION WITH CO2 COMPRESSION AND DEHYDRATION

(71) Applicant: POET Research, Inc., Sioux Falls, SD (US)

(72) Inventor: David Charles Carlson, Brandon, SD (US)

(21) Appl. No.: 19/544,149

(22) Filed: Feb. 19, 2026

Related U.S. Application Data
(60) Provisional application No. 63/763,762, filed on Feb. 26, 2025.

(51) Int. Cl. B01D 53/18; B01D 53/26; C12F 3/04; C12P 7/06

(52) U.S. Cl. CPC B01D 53/18; B01D 53/263; C12F 3/04; C12P 7/06; B01D 2252/2023; B01D 2256/22; B01D 2257/704; B01D 2257/80

(57) ABSTRACT

Systems and methods of integrating gas from a fermentation system with a carbon dioxide compression and dehydration system.

### CROSS-REFERENCE TO RELATED APPLICATION

This nonprovisional patent application claims the benefit of U.S. Provisional Patent Application Ser. No. 63/763,762, filed on Feb. 26, 2025, wherein said provisional patent application is incorporated herein by reference in its entirety.

### BACKGROUND

Carbon dioxide (CO2) capture and sequestration (CCS) is being pursued by many organizations as a means to reduce net CO2 emissions into the atmosphere and address climate change concerns. The CO2 from bioethanol facilities is a particularly attractive feedstock for CCS projects because it is naturally 99%+ pure CO2 on a dry basis and requires minimal cleanup and processing prior to sequestration compared to other sources of CO2 (such as flue gas from coal or natural gas combustion).

In the typical ethanol production process, the carbon dioxide produced by fermentation leaves the fermenters saturated with ethanol and water at a concentration dependent on the concentration of ethanol in the fermenter liquid. This raw carbon dioxide is typically sent to a fermentation scrubber where a counter-current flow of clean water is used to remove the ethanol from the carbon dioxide. The processed carbon dioxide is generally then either emitted to the atmosphere, processed through additional pollution control equipment such as a thermal oxidizer before being emitted to the atmosphere, liquefied and sold as liquid carbon dioxide or dry ice, or more recently compressed and sequestered underground. FIG. 4 illustrates a carbon dioxide system 400 for processing raw carbon dioxide gas 405 from fermenters through a scrubber 410 and regenerative thermal oxidizer (RTO) 420. The raw carbon dioxide gas 405 from all of the fermenters and the beerwell is combined in a common header and sent to a fermentation scrubber 410. The raw carbon dioxide gas 405 leaving the fermenters is in vapor/liquid equilibrium with the contents of the fermenter and depending on the ethanol titer may contain approximately 2.2% v/v ethanol. A carbon dioxide blower may be included before or after the scrubber in order to boost the gas pressure. Fresh water enters the top section of the fermentation scrubber 410 and flows down through mass transfer media (most likely either random packing, structured packing, or trays) while the carbon dioxide enters the bottom section of the fermentation scrubber and flows up through the mass transfer media counter-current to the water flow. Depending on the operating conditions of the scrubber 410 (temperature, fresh water rate, type and amount of mass transfer media, etc. . . . ) the carbon dioxide leaving the top of the scrubber 410 may contain between 10-1,000 ppmv (0.001-0.1 mol %) ethanol. The typical fresh water rate may be between 0.3 and 0.7 pounds of water per pound of carbon dioxide. Depending on air permit requirements and other factors the carbon dioxide leaving the scrubber may be discharged directly to the atmosphere or it may be processed in additional pollution control equipment such as a thermal oxidizer (420) to further reduce the concentration of ethanol and other volatile organic compounds before being discharged to the atmosphere.

Alternatively, the cleaned-up carbon dioxide leaving the scrubber may also be liquefied and sold as either liquid carbon dioxide or dry ice or compressed and sequestered. The ethanol rich water from the bottom of the scrubber that may contain between 3%-7% w/w ethanol is typically processed by the facility to recover the ethanol content as final product.

Sequestering carbon dioxide from fermentation involves compressing the carbon dioxide gas from atmospheric pressure to around 1,000-2,000 psi depending on the transportation pipeline configuration and/or the geologic conditions. The final compressed carbon dioxide must meet several quality specifications, typically including a maximum moisture specification in the range of 10-50 lb/mmscf (pounds of water per million standard cubic feet of gas). A typical carbon dioxide collection and compression system utilizing a triethylene glycol (TEG) dehydration system that receives gas that is first “scrubbed” is shown in FIG. 2. Process 200 includes feeding raw gas 205 from one or more fermenters to a fermentation scrubber 210 to remove ethanol from the raw gas 205 using water in a conventional manner to form scrubbed gas 212, which is fed to at least one compression stage 215 to form a compressed gas 217 that is fed to a triethylene glycol (TEG) dehydration system 200 to remove moisture.

### SUMMARY

According to the present disclosure, gas from a fermentation system can be integrated with a carbon dioxide compression and dehydration system in a manner that permits ethanol separation from a carbon dioxide compression and dehydration system while at the same time reducing the water supply to the scrubber system. In some embodiments, the water supplied to the scrubber system can be reduced and/or turned off. In some embodiments, the scrubber system can be bypassed or simply not installed. FIG. 1 is a non-limiting example of a system/process 100 according to the present disclosure that integrates a carbon dioxide compression and dehydration system 108 with a fermentation system 102 of a corn-to-ethanol biorefinery. Fermentation system 102 produces a fermentation broth 114 and raw gas 116, 118, and 120. The fermentation broth 114 is transferred to an ethanol separation system 106 (e.g., distillation and/or membrane separation) to separate ethanol 130 from the fermentation broth 114 to form stillage 128. Raw gas 116 can be sent to carbon dioxide scrubbing system 104 and/or directly to carbon dioxide compression and dehydration system 108. In some embodiments, raw gas 118 is sent to carbon dioxide scrubbing system 104 to remove ethanol from raw gas 118 and form a liquid 110 that includes the ethanol that is separated (“scrubbed”) from raw gas 118 and form scrubbed gas 112, which is carbon dioxide compression and dehydration system 108. Optionally, or alternatively, raw gas 120 is sent directly to carbon dioxide compression and dehydration system 108. As discussed in more detail below, carbon dioxide compression and dehydration system 108 is configured according to the present disclosure so that one or more liquid streams 124 can be separated during the process of carbon dioxide compression and dehydration system to form compressed carbon dioxide 122 that can be transported and sequestered. Advantageously, by integrating raw gas 116 with a carbon dioxide compression and dehydration system 108 according to the present disclosure one or more liquid streams 124 include ethanol in an amount such that the water supplied to carbon dioxide scrubbing system 104 can be reduced, or even turned off, thereby saving significant amounts of fresh water that would otherwise be supplied to carbon dioxide scrubbing system 104.

Non-limiting examples of dehydration technologies include glycol absorption using triethylene glycol (TEG), desiccant adsorption systems, semi-permeable membrane systems, and refrigeration-based dehydration systems (Joule-Thomson refrigeration and/or mechanical refrigeration).

Both mechanical refrigeration and Joule-Thomson refrigeration systems work by cooling the gas stream below its dew point and removing moisture via condensation.

If a refrigeration-based dehydration system is used, the amount of clean water used in the ethanol plant fermentation scrubber may be significantly reduced and/or eliminated entirely (e.g., turning off the fresh water), resulting in a significant water balance improvement. This would increase the concentration of ethanol in the carbon dioxide feeding the compression system, but because refrigeration-based dehydration systems remove any condensable compounds (such as ethanol) from the gas in addition to water, the concentration of ethanol in the final compressed carbon dioxide may not be significantly different. For example, as shown in FIG. 3, process 300 supplies raw gas 305 from fermentation as feed directly to carbon dioxide compression and refrigeration-based dehydration system 310 so that liquid 315 including water and ethanol can be separated therefrom in a manner that a fermentation scrubber can be avoided.

Both refrigeration-based dehydration systems are potentially susceptible to hydrate formation depending on the operating temperatures of the system. Gas hydrates are solid, ice-like, clathrate compounds that can form at low temperatures from mixtures of water and various gasses such as carbon dioxide and methane and can cause operational issues due to plugging or blocking pipes and other process equipment. One method of preventing hydrate formation is the addition of a thermodynamic hydrate inhibitor (THI) such as methanol or ethanol.

According to another aspect of the present disclosure, the higher concentration of ethanol in the feed gas, resulting from reducing scrubber water flow or eliminating the scrubber entirely, would also act as a thermodynamic hydrate inhibitor (THI) and reduce and/or eliminate the need for a dedicated THI addition. For example, a refrigeration-based dehydration system such as a Joule-Thomson dehydration system could be operated below the hydrate formation temperature without the separate addition of any thermodynamic hydrate inhibitors (such as methanol) due to a higher concentration of ethanol (which acts as a thermodynamic hydrate inhibitor) in the feed carbon dioxide with the scrubber water significantly reduced or off. In some embodiments, the amount of fresh water used in the ethanol plant fermentation scrubber can be adjusted to target a particular ethanol concentration in the liquid purge stream from the Joule-Thomson dehydration system to achieve the necessary hydrate formation temperature reduction and maximize overall ethanol recovery.

### BRIEF DESCRIPTION OF DRAWINGS

Various examples of the present disclosure will be discussed with reference to the appended drawings. These drawings depict only illustrative examples of the disclosure and are not to be considered limiting of its scope.

FIG. 1 is a non-limiting example of a system according to the present disclosure;

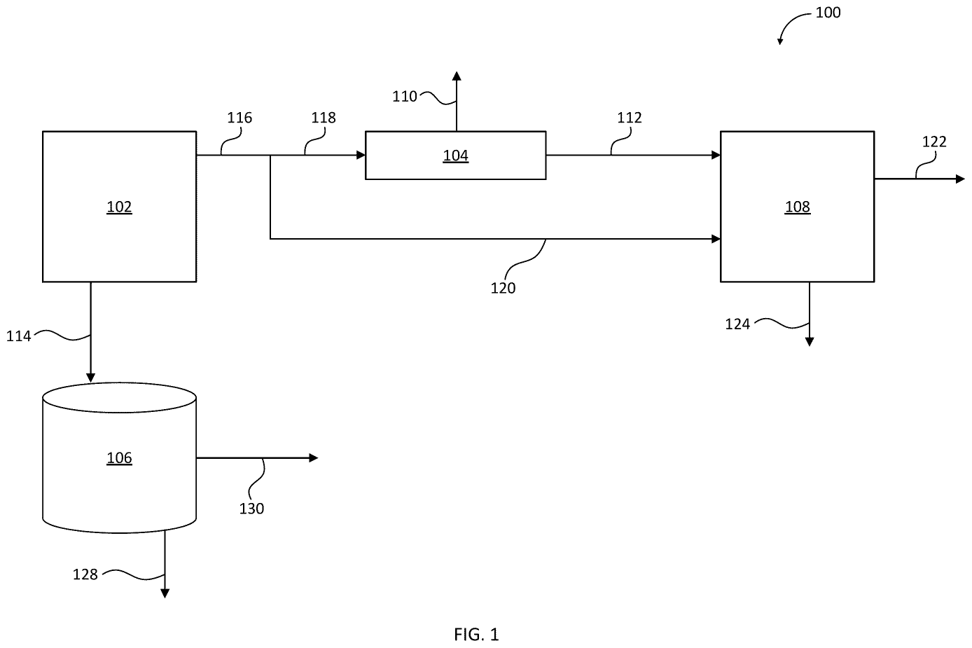

FIG. 2 shows a carbon dioxide collection and compression system utilizing a triethylene glycol (TEG) dehydration system that receives gas that is first “scrubbed”;

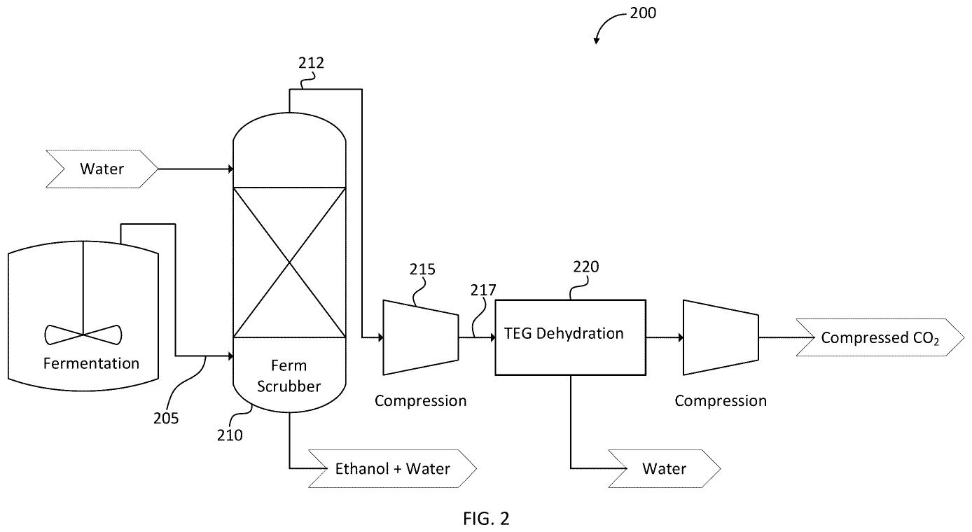

FIG. 3 shows a process that supplies raw gas from fermentation as feed directly to a carbon dioxide compression and refrigeration-based dehydration system;

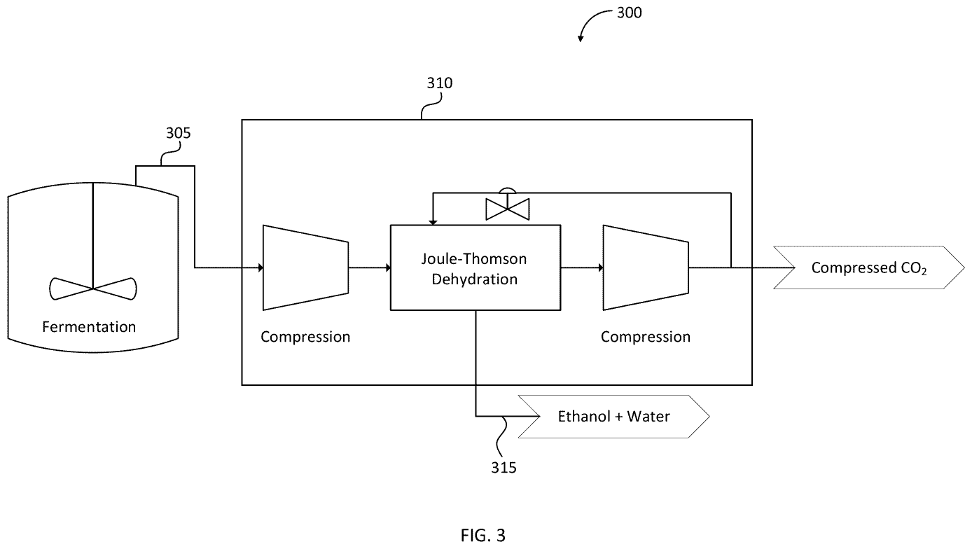

FIG. 4 illustrates a carbon dioxide system for processing raw carbon dioxide gas from fermenters through a scrubber and regenerative thermal oxidizer (RTO);

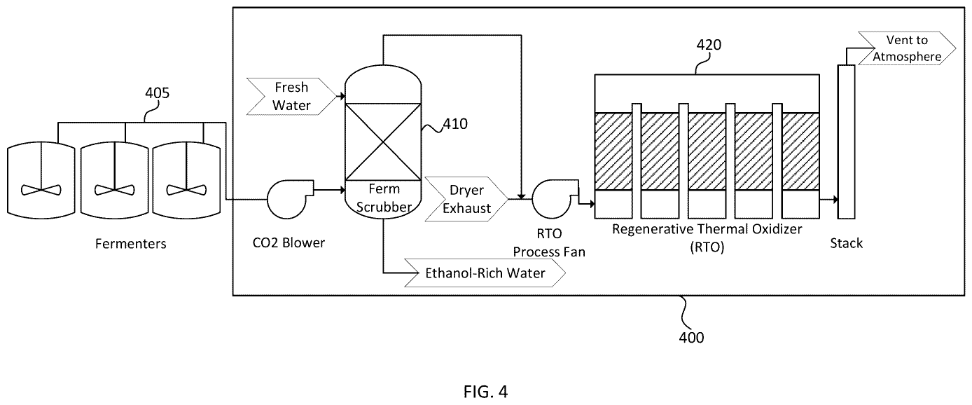

FIG. 5 shows a carbon dioxide compression and dehydration system that includes a multi-stage reciprocating compressor;

FIG. 6 shows an example of a carbon dioxide compression and refrigeration-based dehydration system that includes a mechanical refrigeration dehydration system in a multi-stage reciprocating compressor;

FIG. 7 shows a phase diagram for carbon dioxide overlaid with two example saturated water content lines relative to a “hydrate formation temperature” line;

FIG. 8 illustrates a pressure vs enthalpy diagram;

FIG. 9 illustrates a non-limiting example of a carbon dioxide compression and refrigeration-based dehydration system that includes a Joule-Thomson refrigeration-based dehydration system in a multi-stage reciprocating compressor;

FIG. 10 shows an energy integration scheme in a carbon dioxide compression and refrigeration-based dehydration system;

FIG. 11 shows another energy integration scheme in a carbon dioxide compression and refrigeration-based dehydration system; and

FIG. 12 shows a carbon dioxide compression and refrigeration-based dehydration system discussed in Example 3.

### DETAILED DESCRIPTION

For CCS, the typical pressure to geologically sequester carbon dioxide or transport it on a pipeline ranges from 1,000-2,000 psig. In general, there are pressure drop advantages to operating a pipeline system in the supercritical region of carbon dioxide (above the critical point of 87.8° F. and 1,070 psia). The typical process equipment considered to compress carbon dioxide to the appropriate pressure may include centrifugal fans (such as those manufactured by Twin City Fan or New York Blower), multistage centrifugal blowers (such as those manufactured by Hoffman Lamson or Lone Star Blower), reciprocating compressors (such as those manufactured by Chart Industries or Ariel Corporation), or integrally geared compressors (such as those manufactured by MAN Energy Solutions or Atlas Copco).

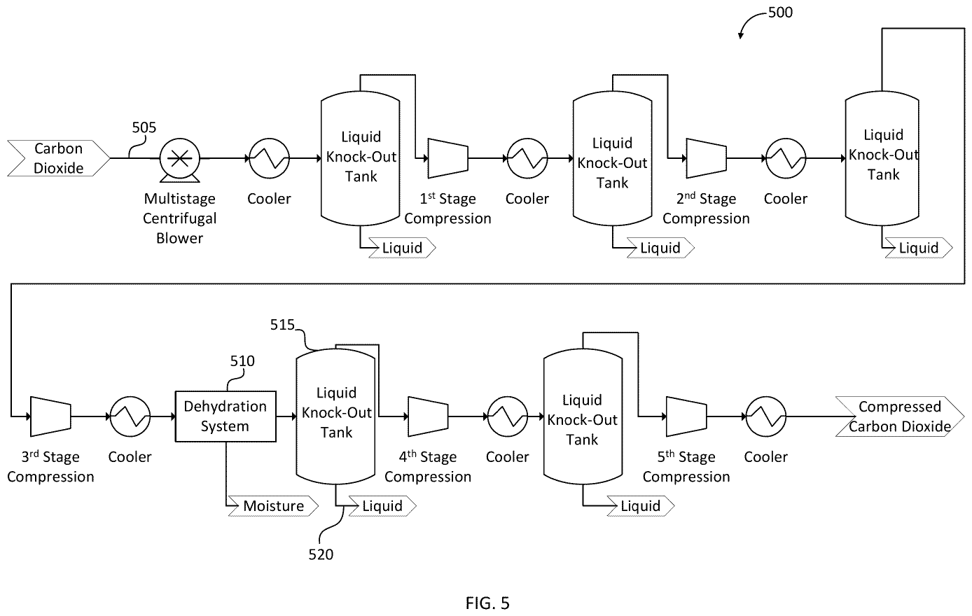

The number of stages in a multi-stage reciprocating compressor can vary and depend on the final discharge pressure. In some embodiments, a multi-stage reciprocating compressor includes at least two stages, at least 3 stages, at least 4 stages, or even at least 5 stages. A non-limiting embodiment of a compression system that includes a multi-stage reciprocating compressor is illustrated in FIG. 5. FIG. 5 shows a carbon dioxide compression and dehydration system 500 that includes a multistage centrifugal blower to boost the pressure from atmospheric to approximately 20-25 psig followed by a five-stage reciprocating compressor to form a compressed gas having a final discharge pressure. The gas is cooled after each stage of compression to remove the heat generated from the compression. Typical cooling systems used include fin/fan air coolers (such as those manufactured by AXH Air-Coolers or Chart Industries) or wet surface air coolers (such as those manufactured by Alfa Laval or Evapco) to transfer the heat to outdoor air or shell and tube heat exchangers to transfer the heat to a circulating cooling media such as cooling water from a cooling tower or a glycol cooling solution.

The gas 505 fed to the multistage centrifugal blower of the carbon dioxide compression and dehydration system 500 is saturated with water from the fermentation scrubber, and because the water saturation content of carbon dioxide decreases with increasing pressure water condenses in each of the cooling steps. For example, in some embodiments, each of the coolers in FIG. 5 can cool the gas to below the dew point except for the coolers after the dehydration system 510. Also, the cooler after the multistage centrifugal blower may not cool below the dew point (likely dew point=~115 F, likely cooling temperature range 80-120 F). Any water that condenses or lube oil carryover from the compressor is removed in a liquid knock-out tank/vessel prior to the next stage of compression in order to prevent any damage to the compressor due to liquid in the inlet gas.

A dehydration system is used after one or more compression stages (e.g., after either the 3rd or 4th compression stage) to remove any remaining moisture down to a typical target specification. The decision of how to order the dehydration system between the compression stages depends on the specific dehydration technology selected and the interstage pressures between all of the compression stages. Typically, the dehydration system will be operated above 300 psig in order to minimize the physical size of the equipment and below the critical pressure of carbon dioxide (1,070 psia) where the phase equilibria is more favorable, particularly for absorption-based processes. For example, referring to FIG. 5, after the 3^{rd} compression stage a dehydration system is used to remove any remaining moisture down to a typical target specification of between 10-50 pounds of water per million standard cubic feet of carbon dioxide.

Common dehydration technology approaches include triethylene glycol (TEG) absorption, desiccant systems, semi-permeable membranes, mechanical refrigeration, and Joule-Thomson refrigeration. A triethylene glycol absorption system utilizes a solution of triethylene glycol to absorb water from the compressed carbon dioxide gas followed by a separate regeneration step to evaporate the water out of the triethylene glycol. A desiccant system utilizes silica gel or molecular sieve zeolites to adsorb water from the carbon dioxide onto a solid media substrate followed by a low-pressure regeneration step to desorb the water from desiccant material. A semi-permeable membrane system utilizes a polymeric membrane that allows water to preferentially diffuse across the membrane surface and into a sweep gas of lower water concentration. In embodiments of the present disclosure that use a refrigeration-based dehydration system as the dehydration system 510, the liquid 520 from the liquid knock-out tank 515 positioned directly after the refrigeration-based dehydration system would include ethanol such that a scrubber system prior to the carbon dioxide compression and dehydration system 500 could be avoided or the water supplied to such a scrubber system could be significantly reduced.

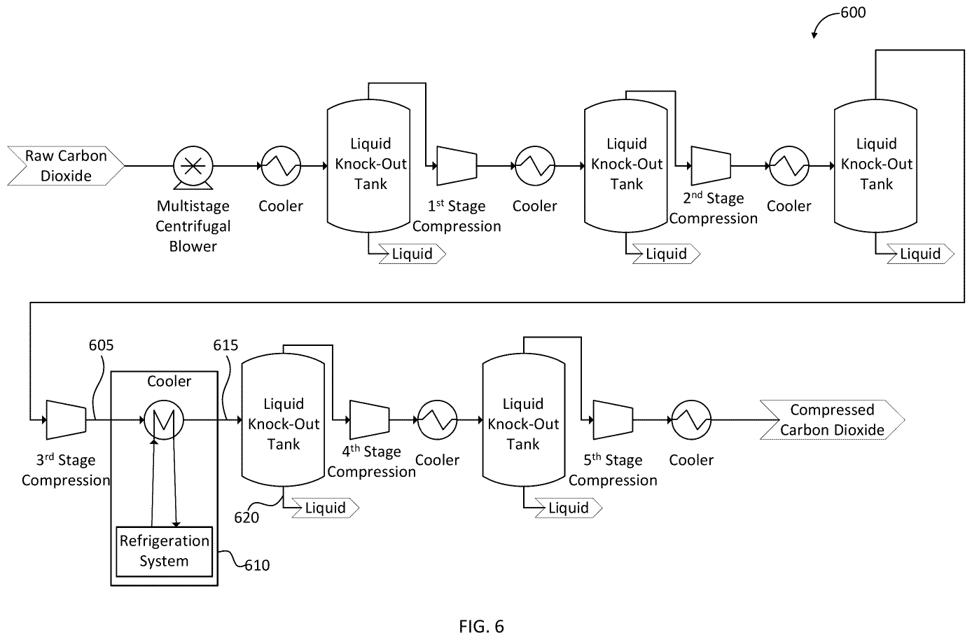

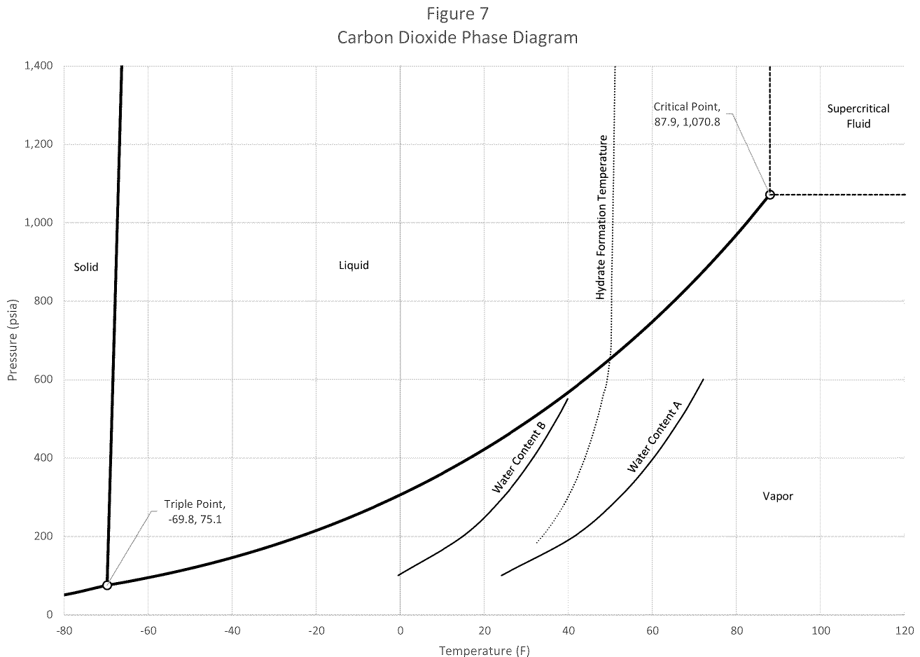

A non-limiting example of a carbon dioxide compression and refrigeration-based dehydration system 600 that includes a mechanical refrigeration dehydration system 610 in a multi-stage reciprocating compressor is shown in FIG. 6. The mechanical refrigeration dehydration system 610 utilizes a refrigeration cycle to cool the compressed carbon dioxide 605 to a temperature lower than what can be consistently achieved using one of the standard cooling methods described above. At a low enough temperature of the carbon dioxide 615, enough of the water condenses out in liquid 620 to meet the target moisture specification. FIG. 7 shows a phase diagram for carbon dioxide overlaid with two example saturated water content lines relative to a “hydrate formation temperature” line; “water content A” line and “water content B” line (discussed further below). Different saturated water content lines can be used to estimate the required temperature at varying pressures in order to reach various moisture levels such as 10, 30, or 50 lb water/million standard cubic feet in the final outlet gas.

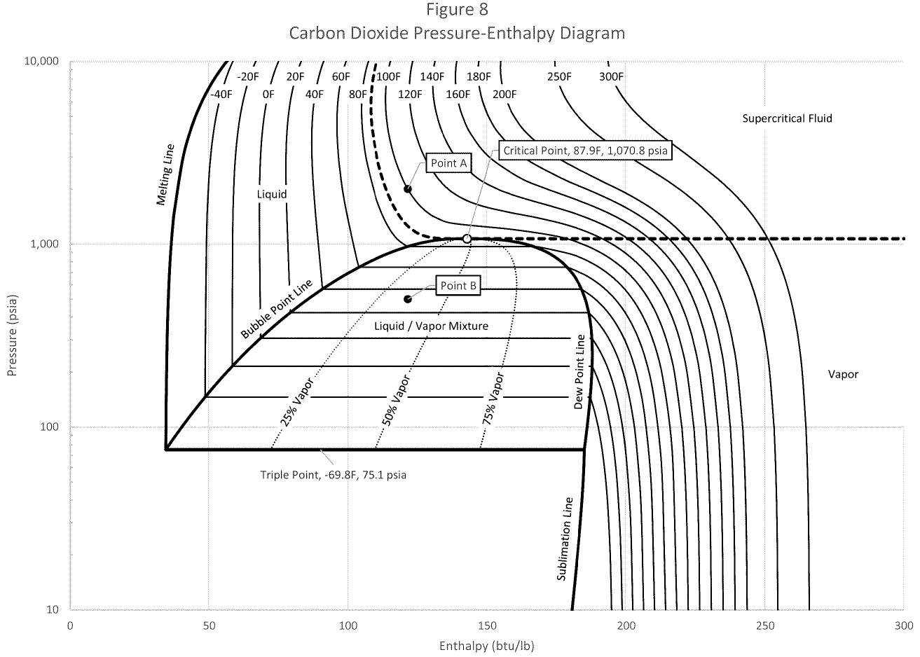

Typical mechanical refrigeration systems use a synthetic refrigerant such as R-134a (1,1,1,2-tetrafluoroethane) or R-410A (mixture of difluoromethane and pentafluoroethane) or natural refrigerants such as ammonia, propane, isobutane, or even carbon dioxide to provide process cooling at temperatures well below ambient by utilizing a refrigeration cycle. A refrigeration cycle generally consists of four basic steps: first compressing a refrigerant from a low pressure to a higher pressure, second cooling the compressed refrigerant to remove the heat of compression, third reducing the pressure of the compressed refrigerant back to the low pressure (typically through a fixed flow orifice or a control valve), and fourth transferring heat from an external source that requires refrigeration to the low-pressure refrigerant. A common thermodynamic property of all refrigerants called the Joule-Thomson effect refers to the decrease in temperature that occurs as the pressure of the refrigerant is reduced. For example, when supercritical carbon dioxide at 2,000 psia and 100° F. is reduced in pressure to 500 psia (such as by flowing across a valve) the temperature decreases to approximately 31° F. and the carbon dioxide is approximately 36% vapor and 64% liquid. This change is illustrated graphically on a pressure vs enthalpy diagram in FIG. 8 with the two conditions described above shown as points A and B respectively. The effects of other similar isenthalpic pressure changes may be estimated in a similar manner.

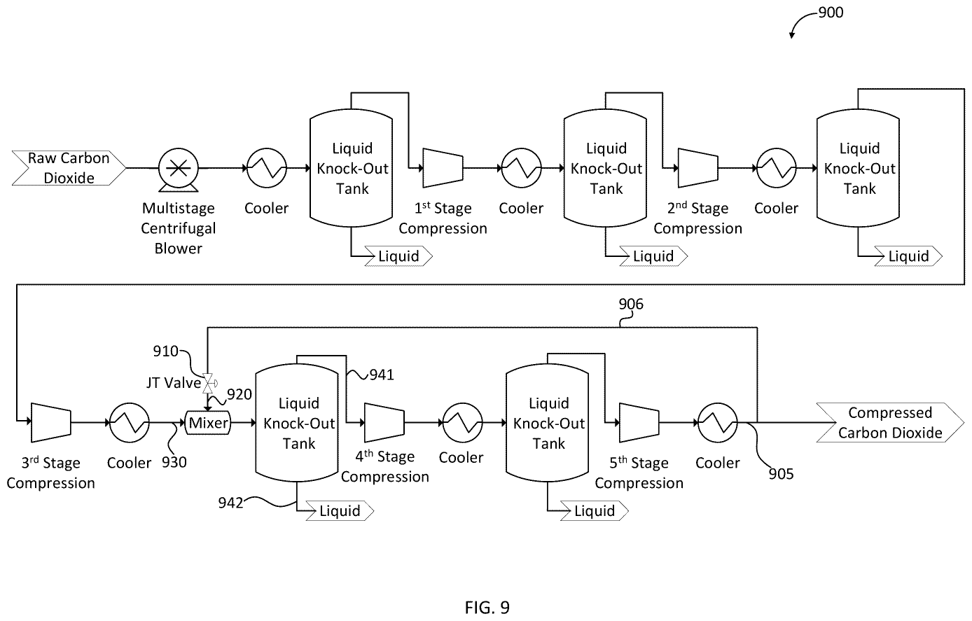

An alternative to a separate mechanical refrigeration unit is to use the refrigerant properties (such as the Joule-Thomson effect) of the already compressed carbon dioxide to cool the carbon dioxide to the target temperature without a separate compressor, cooler, and expansion valve specifically dedicated to a mechanical refrigeration dehydration system like mechanical refrigeration dehydration system 610. In a Joule-Thomson refrigeration-based dehydration system the Joule-Thomson (JT) effect can be used to cool the carbon dioxide to the target temperature. A non-limiting example of a carbon dioxide compression and refrigeration-based dehydration system 900 that includes a Joule-Thomson refrigeration-based dehydration system in a multi-stage reciprocating compressor is shown in FIG. 9. As shown in FIG. 9, a portion 906 of the final compressed carbon dioxide 905 is recycled and throttled across a JT valve 910 to reduce its pressure which cools the carbon dioxide significantly. The cooled carbon dioxide 920 from the JT valve 910 is then mixed in a mixer with cooled carbon dioxide 930 from the previous compression stage (3^{rd} compression stage) and sent to the liquid knock-out tank. Water and ethanol condense due to the low temperature created by the JT effect and the gas going into the 4th stage compression is below the target moisture specification for the final compressed carbon dioxide. Depending on the configuration of the compression system gas from the discharge of either the 4th or 5th compression stages may be sent to the JT valve 910 and mixed with the gas from the discharge of either the 3rd or 4th compression stages.

In order to improve the efficiency of the JT system and reduce the amount of final compressed carbon dioxide sent through the JT valve either or both of the streams feeding the JT system (gas after the previous compression stage cooler 930 going to the mixer or high-pressure gas 910 going to the JT valve) may be pre-cooled by cross exchanging in a heat exchanger with either or both of the streams leaving the liquid knock-out tank (cold gas 941 going into the next compression stage or liquid purge 942 from the knock-out tank).

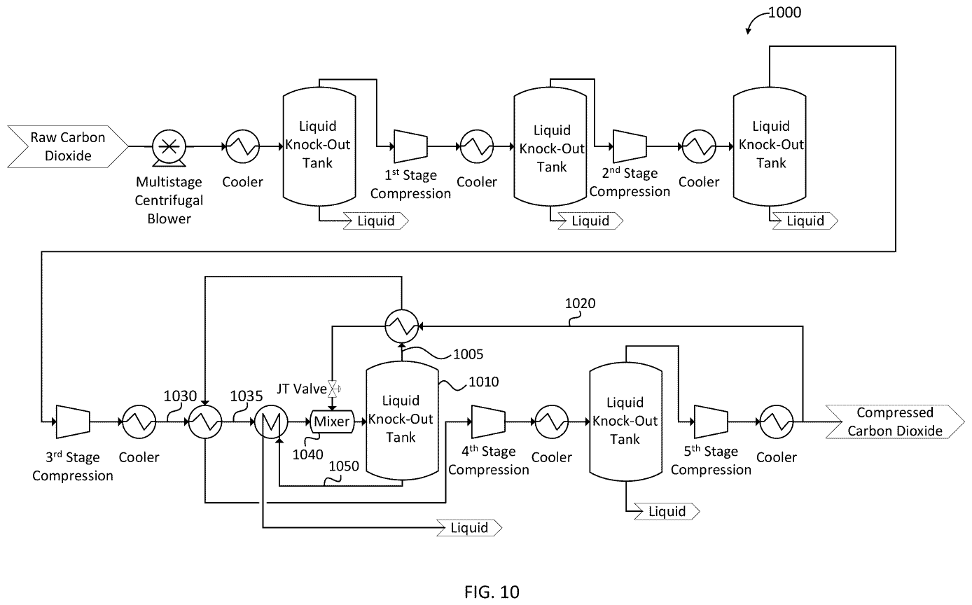

One potential energy integration scheme is shown in carbon dioxide compression and refrigeration-based dehydration system 1000 in FIG. 10 where the cold gas 1005 from the liquid knock-out tank 1010 is first used to pre-cool the high-pressure gas 1020 being recycled to the JT valve and is then used to pre-cool the inlet gas 1030 to the mixer 1040 and the liquid 1050 from the knock-out tank is used to further cool the inlet gas 1035 prior to mixing in the mixer 1040.

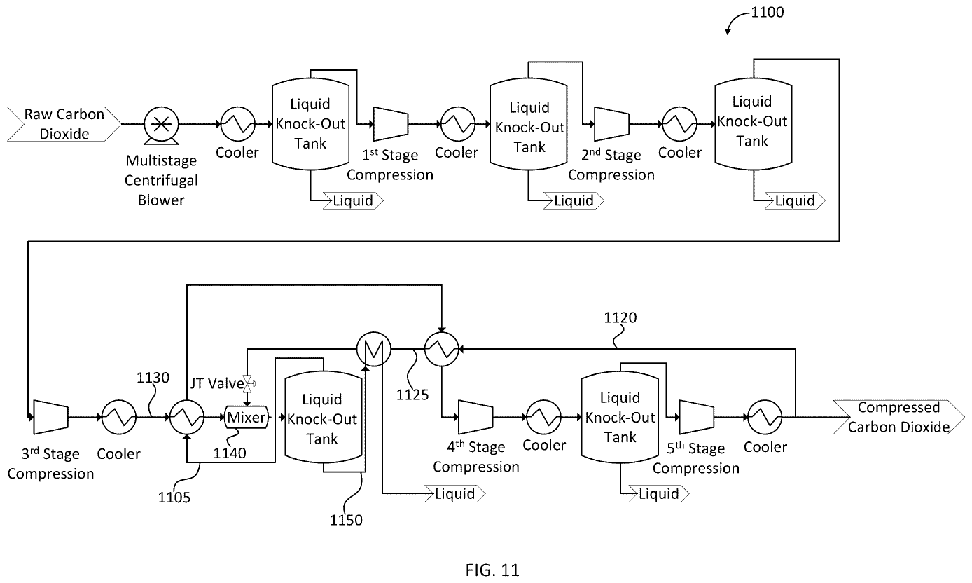

An alternative energy integration scheme is shown in carbon dioxide compression and refrigeration-based dehydration system 1100 in FIG. 11 where the cold gas 1105 from the liquid knock-out tank is first used to pre-cool the inlet gas 1130 to the mixing step in mixer 1140 and is second used to cool the high-pressure gas 1120 being recycled to the JT valve and the liquid 1150 from the knock-out tank is used to further cool the high-pressure gas 1125 immediately before the JT valve. The energy integration scheme shown in FIG. 10 where the high-pressure gas is the first to be pre-cooled is expected to be slightly more energy efficient below operating pressures of approximately 1,400 psia. An example of the JT dehydration process is further described in U.S. Pat. No. 11,125,499 (Mckay et al.).

One of the advantages of utilizing a refrigeration-based dehydration system (e.g., a mechanical refrigeration and/or JT refrigeration dehydration system) as part of the compression process on carbon dioxide from an ethanol production facility is that the refrigeration-based dehydration step also removes any condensable contaminants, such as ethanol, from the carbon dioxide stream in addition to water. Since the dehydration step removes ethanol from the carbon dioxide it may not be necessary to utilize a fermentation scrubber in order to remove ethanol from the carbon dioxide stream prior to the compression step. The removal of the fermentation scrubber step may have a significant impact on the operations of the ethanol production facility by reducing the total amount of fresh water required. A typical ethanol production facility combines various recycle water sources as well as fresh water with ground corn to form a mash or slurry that is sent to the fermentation process. After fermentation is complete the ethanol is removed and purified using a distillation and molecular sieve system and a majority of the residual corn solids are removed, concentrated and dried using centrifugation, evaporation, and drying systems. The remaining water streams from these processing steps, some of which may contain a small amount of residual corn solids, are typically recycled and used as makeup water to mix with the fresh ground corn. Typical ethanol production facilities do not discharge any of these water streams to a water treatment facility and are monitored to determine how much remaining water is generated and how much makeup water is required in order to avoid accumulating excess water. Reducing or eliminating the use of water in the fermentation scrubber not only reduces the amount of fresh water used, but also reduces the total amount of remaining water generated and makes it easier to avoid accumulating excess water.

One of the potential operational concerns of a refrigeration-based dehydration system is the formation of hydrates due to the low temperatures. Gas hydrates are solid, ice-like, clathrate compounds that can form at low temperatures from mixtures of water and various gasses such as carbon dioxide and methane and can cause operational issues due to plugging or blocking pipes and other process equipment. Hydrate equilibrium data for water and carbon dioxide has been published by Adeniyi (Adeniyi 2020) and others. In addition to the saturated water contents, the carbon dioxide phase diagram in FIG. 7 also shows the measured hydrate formation temperature in the presence of liquid water. Operating a refrigeration-based dehydration system at any temperature less than the hydrate formation temperature at the particular pressure is expected to result in hydrate formation in the process equipment. For example, cooling a compressed gas to reduce the water content from water content A line to water content B line may result in undue hydrate formation if hydrate-mitigation steps are not taken. One method of preventing hydrate formation is the addition of a thermodynamic hydrate inhibitor (THI) such as methanol or ethanol which can lower the hydrate formation temperature. However, this may require copious amounts of hydrate inhibitor which may be cost prohibitive.

Another advantage of eliminating the fermentation scrubber (or reducing the water flow to the fermentation scrubber) in embodiments that includes a mechanical refrigeration and/or a JT refrigeration dehydration system) as part of the compression process on carbon dioxide from an ethanol production facility is that a higher concentration of ethanol in the feed gas results in higher a concentration of ethanol in the liquid knock-out tank after the refrigeration step. This ethanol acts as a thermodynamic hydrate inhibitor and enables the refrigeration-based dehydration system to operate at temperatures below the normal hydrate formation temperature without the separate addition of a dedicated thermodynamic hydrate inhibitor. This can allow the compression and dehydration system to achieve a lower moisture content due to the reduced temperature and also recover more of the ethanol from the carbon dioxide feed gas.

### EXAMPLES

### Example 1

In order to demonstrate how much ethanol and water would be removed by a typical carbon dioxide compression system with a TEG dehydration system a process simulation model was constructed using ChemCad with the electrolyte Non-Random Two Liquid (NRTL) thermodynamic model. The feed gas stream at 90° F. and 14.84 psia was selected to be representative of either a system without a fermentation scrubber (containing 2.20 mol % ethanol and 4.56 mol % water), a system with a poor-performing fermentation scrubber (0.20 mol % ethanol and 4.68 mol % water), or a system with a typical fermentation scrubber (0.05 mol % ethanol and 4.69 mol % water). The compression system was modeled as a multistage centrifugal blower pressurizing the gas to 34.70 psia followed by the first three of five reciprocating compression stages pressurizing the gas to 76.56 psia, 168.9 psia, and 372.7 psia respectively. The dehydration step occurs after the 3^{rd} reciprocating compression stage, so it is not necessary to model the remaining two compression stages as no compositional changes occur in the carbon dioxide. The gas after each compression stage is cooled to between 60° F. and 125° F. and any condensed liquids are removed after each cooling step. Tables 1, 2, and 3 below show the predicted amounts of ethanol and water remaining in the compressed carbon dioxide going into the TEG dehydration system and the amount of ethanol present in each of the liquid condensate streams from the knock-out tanks for each of the scenarios described above.

TABLE 1
Feed Gas with No Fermentation Scrubber

| | Ethanol in | | | | | Ethanol in Condensate after | | | |
|---|---|---|---|---|---|---|---|---|---|
| | Compressed | | Water in Compressed | | | Compression Stage (mass %) | | | |
| Temperature | Carbon Dioxide | | Carbon Dioxide | | | | Stage | Stage | Stage |
| F. | ppmv | mol % | ppmv | mol % | lb/mmscf | Blower | 1 | 2 | 3 |
| 60 | 1,283 | 0.1283 | 411 | 0.0411 | 19.5 | 45.4 | 73.3 | 81.0 | 84.7 |
| 70 | 1,512 | 0.1512 | 496 | 0.0496 | 23.5 | 40.1 | 72.6 | 80.3 | 84.3 |
| 80 | 2,086 | 0.2086 | 707 | 0.0707 | 33.5 | 32.2 | 70.3 | 79.5 | 83.6 |
| 90 | 2,838 | 0.2838 | 991 | 0.0991 | 47.0 | 23.3 | 65.3 | 78.0 | 82.9 |
| 100 | 3,781 | 0.3781 | 1,389 | 0.1389 | 65.9 | 16.4 | 55.4 | 75.4 | 81.7 |
| 110 | 4,936 | 0.4936 | 1,945 | 0.1945 | 92.3 | 11.8 | 40.9 | 71.4 | 79.9 |
| 120 | 6,326 | 0.6326 | 2,703 | 0.2703 | 128.3 | | 27.7 | 65.0 | 77.5 |
| 125 | 7,102 | 0.7102 | 3,184 | 0.3184 | 151.1 | | 23.1 | 60.1 | 76.0 |

TABLE 2
Feed Gas with Poor Performing Fermentation Scrubber

| | Ethanol in | | | | | Ethanol in Condensate after | | | |
|---|---|---|---|---|---|---|---|---|---|
| | Compressed | | Water in Compressed Carbon | | | Compression Stage (mass %) | | | |
| Temperature | Carbon Dioxide | | Dioxide | | | Blowe | Stage | Stage | Stage |
| F. | ppmv | mol % | ppmv | mol % | lb/mmscf | r | 1 | 2 | 3 |
| 60 | 954 | 0.0954 | 585 | 0.0585 | 27.8 | 2.8 | 6.5 | 17.1 | 53.7 |
| 70 | 1,087 | 0.1087 | 708 | 0.0708 | 33.6 | 2.2 | 5.8 | 14.0 | 47.4 |
| 80 | 1,304 | 0.1304 | 1,032 | 0.1032 | 49.0 | 1.7 | 4.4 | 10.3 | 31.1 |
| 90 | 1,432 | 0.1432 | 1,473 | 0.1473 | 69.9 | 1.4 | 3.4 | 7.7 | 20.2 |
| 100 | 1,529 | 0.152 | 2,055 | 0.2055 | 97.5 | 1.1 | 2.6 | 5.8 | 14.3 |
| | | 9 | | | | | | | |
| 110 | 1,595 | 0.159 | 2,816 | 0.2816 | 133.6 | 0.8 | 2.0 | 4.5 | 10.5 |
| | | 5 | | | | | | | |
| 120 | 1,641 | 0.164 | 3,799 | 0.3799 | 180.3 | | 1.6 | 3.5 | 7.9 |
| | | 1 | | | | | | | |
| 125 | 1,658 | 0.165 | 4,388 | 0.4388 | 208.3 | | 1.4 | 3.1 | 6.9 |
| | | 8 | | | | | | | |

TABLE 3
Feed Gas with Typical Fermentation Scrubber

| | Ethanol in | | | | | Ethanol in Condensate after | | | |
|---|---|---|---|---|---|---|---|---|---|
| | Compressed | | Water in Compressed Carbon | | | Compression Stage (mass %) | | | |
| Temperature | Carbon Dioxide | | Dioxide | | | | Stage | Stage | Stage |
| F. | ppmv | mol % | ppmv | mol % | lb/mmscf | Blower | 1 | 2 | 3 |
| 70 | 361 | 0.0361 | 938 | 0.0938 | 44.5 | 0.5 | 1.1 | 2.5 | 5.5 |
| 80 | 380 | 0.0380 | 1,319 | 0.1319 | 62.6 | 0.4 | 0.9 | 1.9 | 4.2 |
| 90 | 394 | 0.0394 | 1,827 | 0.1827 | 86.7 | 0.3 | 0.7 | 1.5 | 3.2 |
| 100 | 404 | 0.0404 | 2,496 | 0.2496 | 118.5 | 0.3 | 0.6 | 1.2 | 2.5 |
| 110 | 410 | 0.0410 | 3,364 | 0.3364 | 159.7 | 0.2 | 0.4 | 0.9 | 1.9 |
| 120 | 416 | 0.0416 | 4,479 | 0.4479 | 212.6 | | 0.3 | 0.7 | 1.5 |
| 125 | 421 | 0.0421 | 5,145 | 0.5145 | 244.2 | | 0.3 | 0.7 | 1.4 |

### Example 2

In order to demonstrate the effectiveness of a refrigeration-based dehydration system for removing ethanol from a stream of compressed carbon dioxide a process simulation model was constructed using ChemCad with the electrolyte Non-Random Two Liquid (NRTL) thermodynamic model. The feed gas stream at 90° F. and 14.84 psia was selected to be representative of operating the system without a fermentation scrubber and was comprised of 2.20 mol % ethanol, 4.56 mol % water, and the balance carbon dioxide. The compression system was modeled as a multistage centrifugal blower pressurizing the gas to 34.70 psia followed by the first three of five reciprocating compression stages pressurizing the gas to 76.56 psia, 168.9 psia, and 372.7 psia respectively. The dehydration step occurs after the 3^{rd} reciprocating compression stage, so it is not necessary to model the remaining two compression stages as no compositional changes occur in the carbon dioxide. The gas is cooled to 125° F. after the first three stage of compression (multistage blower and the 1^{st} and 2^{nd} reciprocating stages) and to between 10° F. and 80° F. after the third reciprocating stage and any condensed liquids are removed after each cooling step. Table 4 shows the impact of dehydration temperature on the ethanol and water composition of the compressed carbon dioxide and the ethanol composition of the condensed liquid leaving after the dehydration step. These results indicate that at a temperature of between 20° F. and 50° F. the refrigeration dehydration system is able to achieve similar levels of ethanol in the final carbon dioxide stream as a counter-current fermentation scrubber

TABLE 4

| | Ethanol in | | | | | Ethanol in |
|---|---|---|---|---|---|---|
| | Compressed | | Water in Compressed | | | Refrigerant Stage |
| Temperature | Carbon Dioxide | | Carbon Dioxide | | | Purge Liquid |
| F. | ppmv | mol % | ppmv | mol % | 1b/mmscf | mass % |
| 20 | 255 | 0.0255 | 105 | 0.0105 | 5.0 | 78.5 |
| 30 | 384 | 0.0384 | 159 | 0.0159 | 7.5 | 78.4 |
| 40 | 565 | 0.0565 | 235 | 0.0235 | 11.1 | 78.2 |
| 50 | 818 | 0.0818 | 342 | 0.0342 | 16.2 | 78.0 |
| 60 | 1,164 | 0.1164 | 490 | 0.0490 | 23.2 | 77.8 |
| 70 | 1,628 | 0.1628 | 692 | 0.0692 | 32.8 | 77.5 |
| 80 | 2,241 | 0.2241 | 965 | 0.0965 | 45.8 | 77.1 |

### Example 3

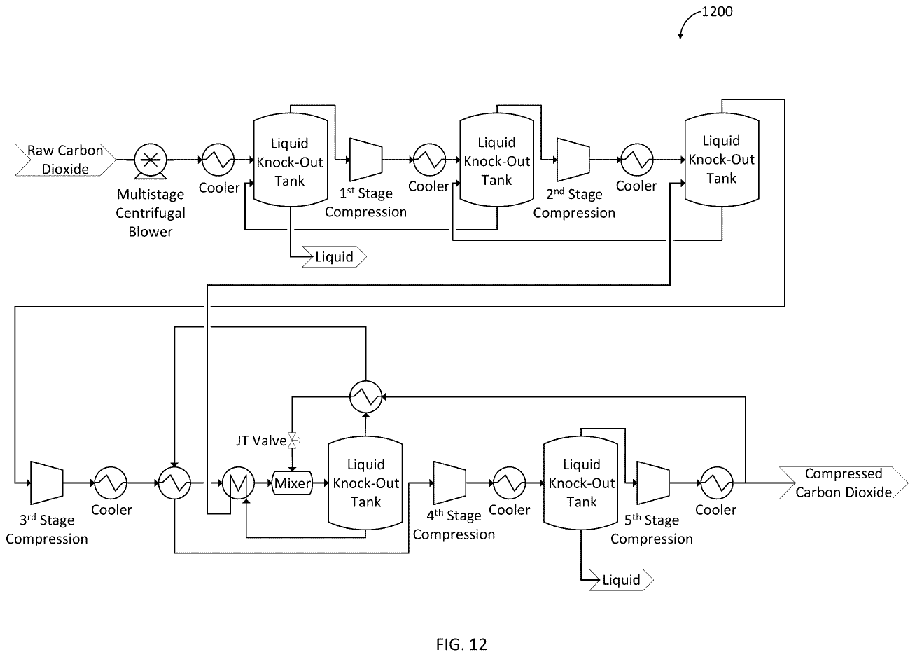

A similar process simulation model was constructed as in example 2, however in this example the liquid purge from each stage where a significant amount of condensate would be expected was returned to the previous stage liquid knock-out tank as shown in the carbon dioxide compression and refrigeration-based dehydration system 1200 in FIG. 12. As the results in Table 5 show, this results in a slightly higher ethanol concentration and a slightly lower water concentration in each stream as compared to the original model in example 1.

TABLE 5

| | Ethanol in | | | | | Ethanol in |
|---|---|---|---|---|---|---|
| | Compressed | | Water in Compressed | | | Refrigerant Stage |
| Temperature | Carbon Dioxide | | Carbon Dioxide | | | Purge Liquid |
| F. | ppmv | mol % | ppmv | mol % | lb/mmscf | mass % |
| 20 | 299 | 0.0299 | 75 | 0.0075 | 3.5 | 88.8 |
| 30 | 449 | 0.0449 | 113 | 0.0113 | 5.4 | 88.7 |
| 40 | 662 | 0.0662 | 168 | 0.0168 | 8.0 | 88.6 |
| 50 | 957 | 0.0957 | 244 | 0.0244 | 11.6 | 88.5 |
| 60 | 1,362 | 0.1362 | 351 | 0.0351 | 16.7 | 88.3 |
| 70 | 1,906 | 0.1906 | 498 | 0.0498 | 23.6 | 88.1 |
| 80 | 2,626 | 0.2626 | 698 | 0.0698 | 33.1 | 87.8 |

### Example 4

A similar process simulation model was constructed as in example 2, however in this example the feed gas was selected to be representative of operations with a normal fermentation scrubber and consisted of 500 ppmv (0.05 mol %) ethanol, 4.69 mol % water, and the balance carbon dioxide. As the results in Table 6 show, the concentrations of ethanol are significantly less and the moisture contents are slightly higher at the same temperature as compared to the previous two examples. The ethanol content of the purge liquid may provide a slight thermodynamic hydrate inhibitor effect, but not as significant compared to either of the previous examples with no fermentation scrubber.

TABLE 6

| | Ethanol in | | | | | Ethanol in |
|---|---|---|---|---|---|---|
| | Compressed | | Water in Compressed | | | Refrigerant Stage |
| Temperature | Carbon Dioxide | | Carbon Dioxide | | | Purge Liquid |
| F. | ppmv | mol % | ppmv | mol % | lb/mmscf | mass % |
| 20 | 88 | 0.0088 | 135 | 0.0135 | 6.4 | 7.1 |
| 30 | 118 | 0.0118 | 207 | 0.0207 | 9.8 | 6.7 |
| 40 | 154 | 0.0154 | 310 | 0.0310 | 14.7 | 6.1 |
| 50 | 194 | 0.0194 | 457 | 0.0457 | 21.7 | 5.5 |
| 60 | 235 | 0.0235 | 662 | 0.0662 | 31.4 | 4.8 |
| 70 | 276 | 0.0276 | 943 | 0.0943 | 44.8 | 4.0 |
| 80 | 313 | 0.0313 | 1,323 | 0.1323 | 62.8 | 3.4 |

### Example 5

In order to determine the most efficient method of interchanging heat with a Joule-Thomson dehydration system a process simulation model was constructed using the equation-of-state and thermodynamics for carbon dioxide proposed by Span and Wagner. Carbon dioxide was the only component considered in order to simplify the model calculations. The feed gas stream was assumed to be 90° F. and 4″ WC pressure (14.84 psia). A multistage centrifugal blower was assumed to first compress the gas to 20 psig (34.70 psia) at an isentropic efficiency of 70%. After the multistage centrifugal blower, a reciprocating compressor was assumed to compress the carbon dioxide to the final pressure in five stages of equal compression ratio with each stage operating at an isentropic efficiency of 70%. The gas was cooled to 125° F. after each of the compression steps. Final compressed gas was recycled to a Joule-Thomson dehydration system operating at 50° F. and configured after the third reciprocating compression stage. Two different energy integration schemes were modeled where the cold gas from the Joule-Thomson dehydration step was either used to cool the high-pressure gas first and the raw gas second (corresponding to FIG. 10) or to cool the raw gas first and the high-pressure gas second (corresponding to FIG. 11). In both of the two energy integration schemes the minimum approach temperature of the interchangers (temperature of the hot stream entering minus the temperature of the cold stream leaving or the temperature of the hot stream leaving minus the temperature of the cold stream entering) was 15° F. A summary of the compression energy used in a system utilizing a TEG dehydration system (no Joule-Thomson dehydration system), a Joule-Thomson dehydration system with no energy integration, and Joule-Thomson dehydration systems with either of the energy integration schemes described above is shown in table 7 below. According to this analysis using the cold gas from the Joule-Thomson dehydration system to cool the high-pressure gas first and the raw gas second (corresponding to FIG. 10) is slightly more energy efficient at final discharge pressures below 1,400 psig and essentially the same efficiency at final discharge pressures above 1,400 psig.

TABLE 7

| | Total Compression Energy (kWh | | | |
|---|---|---|---|---|
| | per metric tonne of carbon dioxide) | | | |
| | | | | JT |
| | | | JT | Dehydration |
| | | JT | Dehydration | with Cold |
| Final | | Dehydration | with Cold | Gas Used to |
| Discharge | TEG | with no | Gas Used to | Cool High- |
| Pressure | Dehydration | Energy | Cool Raw | Pressure |
| (psig) | System | Integration | Gas First | Gas First |
| 1,000 | 104.7 | 175.9 | 112.2 | 109.5 |
| 1,100 | 106.8 | 151.4 | 112.7 | 111.1 |
| 1,200 | 108.8 | 139.6 | 113.3 | 112.6 |
| 1,300 | 110.6 | 132.7 | 114.0 | 113.8 |
| 1,400 | 112.2 | 128.4 | 114.8 | 114.8 |
| 1,500 | 113.6 | 125.8 | 115.7 | 115.8 |
| 1,600 | 115.0 | 124.6 | 116.7 | 116.8 |
| 1,700 | 116.2 | 124.5 | 117.8 | 117.8 |
| 1,800 | 117.4 | 124.9 | 118.9 | 118.9 |
| 1,900 | 118.4 | 125.6 | 119.9 | 119.9 |
| 2,000 | 119.4 | 126.3 | 120.8 | 120.8 |

The following are non-limiting embodiments of the present disclosure:

Embodiment 1. A method comprising:
supplying a feed (raw or scrubbed) gas to a carbon dioxide compression and dehydration process, wherein the feed gas comprises carbon dioxide, ethanol, and moisture, wherein the feed gas is derived from fermenting a fermentable composition; and
separating a liquid from the carbon dioxide compression and dehydration process, wherein the liquid comprises ethanol, wherein the ethanol is present in an amount of at least 10% by weight of the liquid (e.g., at least 20%, at least 30%, at least 50%, at least 60%, at least 70%, at least 80%, or even at least 90% by weight of the liquid).

Embodiment 2. The method of embodiment 1, wherein separating a liquid from the carbon dioxide compression and refrigeration-based dehydration process comprises:
compressing the feed gas to form a compressed gas having a pressure of at least 300 psig (e.g., at least 400 psig, or even at least 500 psig);
cooling the compressed gas to condense at least a portion of water and ethanol present in the compressed gas to form the liquid, and
separating the liquid from the compressed gas.

Embodiment 3. A system comprising:
a fermentation system configured to ferment a fermentable composition and produce a feed (scrubbed or raw) gas comprising ethanol, carbon dioxide, and moisture;
a carbon dioxide compression and dehydration system in fluid communication with the fermentation system to receive the feed gas, wherein the carbon dioxide compression and refrigeration-based dehydration system is configured to discharge a liquid derived at least in part from the feed gas, wherein the liquid comprises ethanol, wherein the ethanol is present in an amount of at least 10% by weight of the liquid (e.g., at least 20%, at least 30%, at least 50%, at least 60%, at least 70%, at least 80%, or even at least 90% by weight of the liquid).

Embodiment 4. A method comprising:
supplying a feed gas (scrubbed or raw) to a carbon dioxide compression and refrigeration-based dehydration process, wherein the feed gas comprises carbon dioxide, ethanol, and moisture, wherein the feed gas is derived from fermenting a fermentable composition, wherein the feed gas has a concentration of ethanol of more than 0.2 mole percent, more than 0.4 mole percent, more than 0.5 mole percent, more than 1 mole percent, more than 1.5 mole percent, or even more than 2.0 mole percent;
compressing the feed gas, or a gas derived from the feed gas, to form a compressed gas having a pressure of at least 500 psig; and
supplying the compressed gas to a refrigeration-based dehydration system.

Embodiment 5. The method of embodiment 3, further comprising separating a liquid from the refrigeration-based dehydration system and/or downstream from the refrigeration-based dehydration system, wherein the liquid comprises ethanol.

Embodiment 6. A system comprising:
a fermentation system configured to ferment a fermentable composition and produce a feed gas (scrubbed or raw) comprising ethanol, carbon dioxide, and moisture;
a carbon dioxide compression and refrigeration-based dehydration system in fluid communication with the fermentation system to receive the feed gas, wherein the feed gas has a concentration of ethanol of more than 0.2 mole percent, more than 0.4 mole percent, more than 0.5 mole percent, more than 1 mole percent, more than 1.5 mole percent, or even more than 1.5 mole percent, and wherein the carbon dioxide compression and dehydration system comprises:
   at least compressor configured to compress the feed gas, or a gas derived from the feed gas, to form a compressed gas having a pressure of at least 500 psig; and
   at least one refrigeration-based dehydration system in fluid communication with the compressed gas.

Embodiment 7. A method comprising:
supplying a raw gas to a carbon dioxide scrubbing process to form a scrubbed gas, wherein the raw gas comprises carbon dioxide, ethanol, and moisture, wherein the raw gas is derived from fermenting a fermentable composition, and wherein the scrubbed gas comprises a target ethanol concentration;
supplying the scrubbed gas to a carbon dioxide compression and refrigeration-based dehydration process,
compressing the scrubbed gas, or a gas derived from the scrubbed gas, to form a compressed gas,
supplying the compressed gas to a refrigeration-based dehydration system, wherein the compressed gas comprises ethanol, and wherein the target ethanol concentration is based on a thermodynamic hydrate inhibitor (THI) value of the ethanol in the compressed gas.

Embodiment 8. The method of embodiment 7, wherein the scrubbing process comprises supplying the raw gas to a water contacting column with a mass ratio of fresh water to carbon dioxide of less than 0.3 (less than 0.2, less than 0.1, etc. . . . ).

Embodiment 9. The method of embodiment 7, further comprising separating a liquid from the refrigeration-based dehydration system and/or downstream from the refrigeration-based dehydration system, wherein the liquid comprises ethanol.

Embodiment 10. The method of embodiment 7, further comprising separating a liquid from the scrubbing process, wherein the liquid comprises ethanol.

Embodiment 11. A system comprising:
a fermentation system configured to ferment a fermentable composition and produce a raw gas comprising ethanol, carbon dioxide, and moisture;
a carbon dioxide scrubbing system in fluid communication with the raw gas, wherein the scrubbing system is configured to separate at least a portion of the ethanol from the raw gas to form a scrubbed gas, wherein the scrubbed gas comprises a target ethanol concentration;
a carbon dioxide compression and refrigeration-based dehydration system in fluid communication with the fermentation system to receive the feed gas, wherein the carbon dioxide compression and dehydration system comprises:
   at least compressor configured to compress the scrubbed gas, or a gas derived from the scrubbed gas, to form a compressed gas; and
   at least one refrigeration-based dehydration system in fluid communication with the compressed gas, wherein the compressed gas comprises ethanol, and wherein the target ethanol concentration is based on a thermodynamic hydrate inhibitor (THI) value of the ethanol in the compressed gas.

Embodiment 12. A method comprising:
supplying a feed gas (raw) to a carbon dioxide compression and refrigeration-based dehydration process, wherein the feed gas comprises carbon dioxide, ethanol, and moisture, wherein the feed gas is derived from fermenting a fermentable composition;
wherein the feed gas has not been processed in a scrubbing system prior to the compression step;
compressing the feed gas, or a gas derived from the feed gas, to form a compressed gas having a pressure of at least 500 psig; and
supplying the compressed gas to a refrigeration-based dehydration system.

Embodiment 13. A system comprising:
a fermentation system configured to ferment a fermentable composition and produce a feed (raw) comprising ethanol, carbon dioxide, and moisture;
a carbon dioxide compression and refrigeration-based dehydration system in fluid communication with the fermentation system to receive the feed gas, wherein the feed gas has not been processed in a scrubbing system prior to the compression system; wherein the carbon dioxide compression and dehydration system comprises:
   at least compressor configured to compress the feed gas, to form a compressed gas having a pressure of at least 500 psig; and
   at least one refrigeration-based dehydration system in fluid communication with the compressed gas.

Embodiment 14. A method comprising:
supplying a raw gas to a carbon dioxide scrubbing process to form a scrubbed gas, wherein the raw gas comprises carbon dioxide, ethanol, and moisture, wherein the raw gas is derived from fermenting a fermentable composition, and wherein the scrubbing process comprises supplying the raw gas to a water contacting column with a mass ratio of fresh water to carbon dioxide of less than 0.3 (less than 0.2, less than 0.1, etc. . . . );
supplying the scrubbed gas to a carbon dioxide compression and refrigeration-based dehydration process,
compressing the scrubbed gas, or a gas derived from the scrubbed gas, to form a compressed gas having a pressure of at least 500 psig; and
supplying the compressed gas to a refrigeration-based dehydration system.

Embodiment 15. A system comprising:
a fermentation system configured to ferment a fermentable composition and produce a raw gas comprising ethanol, carbon dioxide, and moisture;
a carbon dioxide scrubbing system in fluid communication with the raw gas, wherein the scrubbing system is configured to supply the raw gas to a water contacting column with a mass ratio of fresh water to carbon dioxide of less than 0.3 (less than 0.2, less than 0.1, etc. . . . ) and form a scrubbed gas;
a carbon dioxide compression and refrigeration-based dehydration system in fluid communication with the carbon dioxide scrubbing system to receive the scrubbed gas, wherein the carbon dioxide compression and dehydration system comprises:
   at least compressor configured to compress the scrubbed gas, or a gas derived from the scrubbed gas, to form a compressed gas; and
at least one refrigeration-based dehydration system in fluid communication with the compressed gas.

Embodiment 16. A method comprising:
supplying a feed (raw) gas to a carbon dioxide compression and dehydration process, wherein the feed gas comprises carbon dioxide, ethanol, and moisture, wherein the feed gas is derived from fermenting a fermentable composition, and wherein the feed gas has a concentration of ethanol of more than 0.2 mole percent, more than 0.4 mole percent, more than 0.5 mole percent, more than 1 mole percent, more than 1.5 mole percent, or even more than 2.0 mole percent; and
separating a liquid from the carbon dioxide compression and dehydration process, wherein the liquid comprises ethanol, wherein a concentration of ethanol in the liquid at least 10% by weight of the liquid (e.g., at least 20%, at least 30%, at least 50%, at least 60%, at least 70%, at least 80%, or even at least 90% by weight of the liquid).

Embodiment 17. A system comprising:
a fermentation system configured to ferment a fermentable composition and produce a feed (raw) gas comprising ethanol, carbon dioxide, and moisture, wherein the feed gas has a concentration of ethanol of more than 0.2 mole percent, more than 0.4 mole percent, more than 0.5 mole percent, more than 1 mole percent, more than 1.5 mole percent, or even more than 2.0 mole percent;
a carbon dioxide compression and dehydration system in fluid communication with the fermentation system to receive the feed gas, wherein the carbon dioxide compression and dehydration system is configured to discharge a liquid derived at least in part from the feed gas, wherein the liquid comprises ethanol, wherein a concentration of ethanol in the liquid at least 10% by weight of the liquid (e.g., at least 20%, at least 30%, at least 50%, at least 60%, at least 70%, at least 80%, or even at least 90% by weight of the liquid).

Embodiment 18. A method comprising:
supplying a feed gas (scrubbed or raw) to a carbon dioxide compression and refrigeration-based dehydration process, wherein the feed gas comprises carbon dioxide, ethanol, and moisture, wherein the feed gas is derived from fermenting a fermentable composition,
separating a liquid from the carbon dioxide compression and dehydration process, wherein the liquid comprises ethanol,
wherein the refrigeration-based dehydration process is operated at a temperature below the calculated hydrate formation temperature of carbon dioxide in the presence of liquid water,
and wherein no fluid (e.g., a thermodynamic hydrate inhibitor (THI)) that impacts the hydrate formation temperature is added to the gas.

Embodiment 19. A method comprising:
supplying a feed gas (scrubbed or raw) to a carbon dioxide compression and refrigeration-based dehydration process, wherein the feed gas comprises carbon dioxide, ethanol, and moisture, wherein the feed gas is derived from fermenting a fermentable composition,
separating a liquid from the carbon dioxide compression and dehydration process, wherein the liquid comprises ethanol,
wherein the refrigeration-based dehydration process is operated at a temperature less than 50° F. (e.g. less than 40° F., less than 30° F., less than 20° F., or even less than 10° F.),
and wherein no fluid (e.g., a thermodynamic hydrate inhibitor (THI)) that impacts the hydrate formation temperature is added to the gas.

What is claimed is:

1. A method comprising:
supplying a feed gas to a carbon dioxide compression and dehydration process, wherein the feed gas comprises carbon dioxide, ethanol, and moisture, wherein the feed gas is derived from fermenting a fermentable composition; and
separating a liquid from the carbon dioxide compression and dehydration process, wherein the liquid comprises ethanol, wherein the ethanol is present in an amount of at least 10% by weight of the liquid.

2. The method of claim 1, wherein the ethanol is present in an amount of at least 20% by weight of the liquid.

3. The method of claim 1, wherein the ethanol is present in an amount of at least 30% by weight of the liquid.

4. The method of claim 1, wherein the ethanol is present in an amount of at least 70% by weight of the liquid.

5. The method of claim 1, wherein the ethanol is present in an amount of at least 90% by weight of the liquid.

6. The method of claim 1, wherein separating a liquid from the carbon dioxide compression and refrigeration-based dehydration process comprises:
compressing the feed gas to form a compressed gas having a pressure of at least 300 psig;
cooling the compressed gas to condense at least a portion of water and ethanol present in the compressed gas to form the liquid, and
separating the liquid from the compressed gas.

7. The method of claim 1, wherein the feed gas comprises raw gas or scrubbed gas.

8. A method comprising:
supplying a feed gas to a carbon dioxide compression and refrigeration-based dehydration process, wherein the feed gas comprises carbon dioxide, ethanol, and moisture, wherein the feed gas is derived from fermenting a fermentable composition, wherein the feed gas has a concentration of ethanol of more than 0.2 mole percent;
compressing the feed gas, or a gas derived from the feed gas, to form a compressed gas having a pressure of at least 500 psig; and
supplying the compressed gas to a refrigeration-based dehydration system.

9. The method of claim 8, further comprising separating a liquid from the refrigeration-based dehydration system and/or downstream from the refrigeration-based dehydration system, wherein the liquid comprises ethanol.

10. The method of claim 8, wherein the feed gas has a concentration of ethanol of more than 1 mole percent.

11. The method of claim 8, wherein the feed gas has a concentration of ethanol of more than 2 mole percent.

12. The method of claim 1, wherein the feed gas comprises raw gas or scrubbed gas.

13. A method comprising:
supplying a raw gas to a carbon dioxide scrubbing process to form a scrubbed gas, wherein the raw gas comprises carbon dioxide, ethanol, and moisture, wherein the raw gas is derived from fermenting a fermentable composition, and wherein the scrubbed gas comprises a target ethanol concentration;
supplying the scrubbed gas to a carbon dioxide compression and refrigeration-based dehydration process,
compressing the scrubbed gas, or a gas derived from the scrubbed gas, to form a compressed gas,
supplying the compressed gas to a refrigeration-based dehydration system,
wherein the compressed gas comprises ethanol, and wherein the target ethanol concentration is based on a thermodynamic hydrate inhibitor (THI) value of the ethanol in the compressed gas.

14. The method of claim 13, wherein the scrubbing process comprises supplying the raw gas to a water contacting column with a mass ratio of fresh water to carbon dioxide of less than 0.3.

15. The method of claim 13, wherein the scrubbing process comprises supplying the raw gas to a water contacting column with a mass ratio of fresh water to carbon dioxide of less than 0.2.

16. The method of claim 13, wherein the scrubbing process comprises supplying the raw gas to a water contacting column with a mass ratio of fresh water to carbon dioxide of less than 0.1.

17. The method of claim 13, further comprising separating a liquid from the refrigeration-based dehydration system and/or downstream from the refrigeration-based dehydration system, wherein the liquid comprises ethanol.

18. The method of claim 13, further comprising separating a liquid from the scrubbing process, wherein the liquid comprises ethanol.
